(19) United States
(12) Patent Application Publication
(10) Pub. No.: US 2010/0104149 A1
(43) Pub. Date: Apr. 29, 2010

(54) IMAGING OF A TURBID MEDIUM

(75) Inventors: Tim Nielsen, Hamburg (DE); Thomas Koehler, Norderstedt (DE)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/444,694

(22) PCT Filed: Oct. 8, 2007
(86) PCT No.: PCT/IB07/54081

§ 371 Date: Apr. 8, 2009

(30) Foreign Application Priority Data
Oct. 9, 2006 (EP) 06121948.1

(51) Int. Cl. A61B 6/00; G06K 9/00

(52) U.S. Cl. 382/128; 600/476

(57) ABSTRACT

An imaging system for imaging a turbid medium comprises a radiation source to illuminate an object to be imaged. A detection system to detect radiation from the object to produce a plurality of detected radiation levels at respective positions relative to the object. A distinction is made between (i) a central radiation component having passed mainly through an inner region of the object and (ii) a boundary radiation component having passed mainly through a boundary region of the object. On the basis of a comparison of the central radiation component and the boundary radiation component the optical properties, notably optical scattering and optical absorption are derived. From the detected radiation from the object and the optical properties an image of the interior or the object is reconstructed.

### FIELD OF THE INVENTION

The invention pertains to an imaging system for imaging a turbid medium.

### BACKGROUND OF THE INVENTION

An imaging system for imaging a turbid medium is known from the U.S. Pat. No. 6,956,650.

The known imaging system receives radiation which exits from the medium. From the received radiation and variable calibration factors one or more optical properties of the medium are derived. With the use of these optical properties a distribution of the optical properties in the medium is determined.

### SUMMARY OF THE INVENTION

An object of the invention is to provide an imaging system for imaging a turbid medium in which image quality is improved.

This object is achieved by an imaging system for imaging a turbid medium comprising
a radiation source to illuminate an object to be imaged,
a detection system to detect radiation from the object to produce a plurality of detected radiation levels at respective positions relative to the object
the radiation from the object having
a central radiation component having passed mainly through an inner region of the object and
a boundary radiation component having passed mainly through a boundary region of the object,
the imaging system further comprising
an analysis module to derive optical properties of the object on the basis of a comparison of the central radiation component and the boundary radiation component and

a reconstruction module to access the detected radiation levels and the optical properties and reconstruct an image dataset on the basis of the detected radiation levels and the optical properties.

According to the invention, accurate values are obtained for the optical properties of the object, i.e. the turbid medium, to be imaged. On the basis of these accurate values for the optical properties propagation of radiation, notably near-infrared light, through the turbid medium can be modelled, e.g. on the basis of a radiation diffusion model. The radiation detection system measures the radiation level, i.e. the intensity of radiation, notably near-infrared light, that emerges from the object at respective detection positions. On the basis of the modelled propagation of radiation the reconstruction of the image on the basis of the detected radiation levels is adapted to the optical properties of the turbid medium. The adapted reconstruction produces images having a very good diagnostic image quality, that is small details having low contrast are well distinguished in the image. Accordingly, visibility in the reconstructed image of small lesions in the breast being imaged is achieved.

An insight of the present invention is that radiation which propagates more closely to the boundary of the object is more affected by differences between the optical properties of the object and the optical properties that prevail outside of the object. Variation of the optical properties such as optical scattering and optical absorption over a distance that is comparable to the scattering length acts effectively as a boundary. In a turbid medium in which optical scattering dominates of optical absorption, propagation of radiation is well approximated by diffusion of radiation. The diffusion is determined by both optical absorption and optical scattering. The presence of the boundary is essentially determined by the optical scattering. Accordingly, the presence of the boundary allows to independently determine the values of the optical absorption and optical scattering at issue. The boundary radiation component involves radiation which propagates closer to the boundary of the object. The central radiation component involves radiation that propagates mainly through the centre region, far from the boundary, of the object. The optical properties, in particular absorption and scattering, of the object to be imaged can be determined independently by comparing the central radiation component and the boundary radiation component. Radiation that propagates mainly through the centre region, far from the boundary, of the object is affected by both the optical properties, viz optical absorption as well as optical scattering. The independent determination of optical absorption and scattering properties is attributed to the insight that radiation that passes more closely to the boundary of the object is relatively more affected by scattering while radiation passing mainly through the interior of the object is relatively more affected by both absorption and scattering

Because the reconstruction is adapted to the actual values of the optical properties of the object to be imaged, the image quality of images of different objects is less sensitive to differences between the optical properties of individual objects. Accordingly, the image quality obtained is quite insensitive to variations between breasts of different individual patients, so that good image quality is achieved for a very wide class of patients to be examined and image quality is equally good for each individual patient.

These and other aspects of the invention will be further elaborated with reference to the embodiments defined in the dependent Claims.

According to a further aspect of the invention a reference dataset is derived on the basis of the actual optical properties and which represents the way radiation, notably near-infrared radiation, propagates through a homogeneous object having the actual optical properties. In particular the reference dataset represents radiation levels that are detected at respective orientations of radiation having passed through the homogeneous object. The measurement dataset represents the radiation levels which are detected at respective orientations relative to the object to be imaged. Then the image is reconstructed on the basis of the reference dataset and the measurement dataset. Because the reference dataset is adapted to the optical properties of the object to be imaged, the measurement dataset represent relatively small perturbations as compared to the reference dataset. Hence, notably reconstruction on the basis of a so-called Rytov approximation yields good image quality of the reconstructed image. Notably, the reconstruction can be based on ratios of data values of the measurement dataset to corresponding data values of the reference dataset. This renders the reconstruction insensitive to system dependent calibration factors.

According to another aspect of the invention the values of the optical properties of the object to be examined are derived from a comparison between the dependence of the detected radiation levels and simulated radiation levels for pre-determined values of the optical properties on the distance between the source position and the detection position. To this end a large dataset is generated of simulated radiation levels that occur at the various detection positions for a variation of optical properties, notably absorption and scattering within a physiological relevant range and for a plurality of detection positions. On the basis of optimum agreement between the dependence on the distance between the source position and the detection position for the detected and simulated radiation levels, respectively, the actual values of the optical properties of the object to be examined, notably a woman's breast to be imaged, are derived. The optical absorption and optical scattering can be separately derived from the fit to the simulations because in the diffusive regime of optical propagation in the propagation in bulk is driven by both scattering and absorption, while the boundary conditions that apply because the turbid medium is finite (and thus has a boundary) are effectively determined by the optical scattering properties. Accordingly, the comparison implicitly involves a comparison between propagation through the centre of the medium and propagation near the boundary. It appears that the presence of the boundary affects the optical propagation over a distance of a few mean scattering lengths. The comparison between the dependence on the distance between the source position and the detection position for the detected and simulated radiation levels is practically carried out on the basis of a pattern matching procedure. The pattern matching procedure is found to be more stable when as independent co-ordinates k₀=\sqrt{3μₜμₐ} and μₜ are used in stead of the optical properties μₐ (optical absorption coefficient) and μₜ (optical scattering coefficient). Further, the detected radiation levels as well as the simulated radiation levels may be averaged over rotational symmetry of the object. This reduces the amount of data, improves signal-to-noise of the detected radiation levels, while accurate values of the optical properties of the object to be imaged are obtained. More accurate and reliable values for the optical properties of the turbid medium of the object are obtained when also the reflectivity of an inner wall of an examination space in which the object to be examined is positioned. For example, the examination space may be formed as a cup-shaped receptacle in which a woman's breast to be imaged can be placed.

According to a further aspect of the invention the central radiation component and the boundary radiation component are separated on the basis of a separation value of the distance between the position where the radiation source illuminates the object and the position where the detection system detects the radiation from the object. Radiation levels which are detected at a distance between source and detection position less than the separation value concern propagation trajectories that are mainly close to the boundary. Hence, the radiation levels detected at positions closer to the source position than the separation value are assigned to the boundary radiation component. Radiation levels which are detected at a distance between source and detection position larger than the separation value concern propagation trajectories that mainly pass through the central region of the object. Hence, the radiation levels detected at positions closer to the source position than the separation value are assigned to the central radiation component.

The separation value can be set by the user, e.g. on the basis of previous experience. An accurate value of the separation value is estimated on the basis of the mean scattering length of the turbid medium of the object. Notably, the separation value is in the order of one or a few mean scattering lengths. Also in the estimate, the geometry of the perimeter of the object is taken into account which determines the distance of propagation trajectories to the boundary. In a simple spherical or ellipsoidal geometry the average distance of straight propagation paths to the perimeter is the distance to the perimeter from the chord running from a source to a detection position on the perimeter. More accurate estimates are obtained by also taking into account a somewhat banana-shaped spatial distribution of propagation paths. Alternatively, the separation value can be derived from the dependence of the detected radiation levels on the distance between source position and detection position. To this end a pattern analysis may be applied to the graph of the detected radiation levels dependent on the distance between source position and detection position An accurate value of the separation value is obtained from the dependence of the product of the detected radiation level and the distance between source position and detection position on this distance.

According to another aspect of the invention, the imaging system is provided with a fluid dispensing system which provides the fluid that surrounds the object to be imaged. The fluid dispensing system receives the values for the optical properties of the object, notably the woman's breast, to be imaged. On the basis of these values of the optical properties, the matching fluid is prepared so that the optical properties of the matching fluid accurately correspond to the optical properties of the object. The matching fluid may be prepared by mixing or selecting of several basic fluids or by adding particular additives, such as dyes to a basic fluid. In particular examples of a matching fluid are a mixture of milk, water and (black) ink, a mixture of intralipid and water or a mixture of water, Titanium-dioxide, lipids, tensides and various dyes. Accurate correspondence of the optical properties of the matching fluid with the optical properties of the object to be imaged reduces distortions at the surface of the object. Moreover, the accurate correspondence of the optical properties of the breast and the surrounding matching fluid provides a situation in which the Rytov approximation is a very fair approximation and thus leads to a good image quality of the reconstructed image of the interior of the object.

The invention also relates to an assessment combination of a detection system and analysis unit as a separate entity that does not require imaging functionality. This combination derives the values of the optical properties of an object from the detected radiation levels. This is achieved on the basis of the modelled propagation of radiation the reconstruction of the image on the basis of the detected radiation levels is adapted to the optical properties of the turbid medium. These values of the optical properties may be used to make an accurate estimate of the composition of the matching fluid. For this estimate no imaging function is required. The assessment combination of the detection system and analysis unit may be designed as a hand-held unit which may be employed to obtain the actual values of the optical properties of the turbid medium of the object to be examined. Accordingly, the assessment combination of the detection system and analysis unit may be employed to obtain the optical properties of a woman's breast before she is positioned to have the breast imaged by the imaging system of the invention. Further, the assessment combination may be formed as a hand held detection unit which communicates via a cable connection or wireless with the analysis unit. In this way the analysis unit may be shared between the assessment combination and the imaging system.

The invention further relates to a method of imaging a turbid medium. The method of the invention is defined in Claim 7. The method of the invention achieves to reconstruct an image of the interior of the turbid medium. Perturbations and/or artefacts due to variations of the optical properties of the object(s) to be imaged are avoided. In particular the method of the invention may be employed in optical mammography where a woman's breast is examined by way of near-infrared radiation. In the field of optical mammography, the method of the invention provides images of a high diagnostic quality, i.e. where small details with low contrast are rendered well visible.

The invention also pertains to a computer program as defined in Claim 8. De computer program of the invention can be provided on a data carrier such as a CD-rom disk, or the computer programme of the invention can be downloaded from a data network such as the world-wide web. When installed in the computer included in an imaging system the imaging system is enabled to operate according to the invention and achieve images with low artefact level of the interior of a turbid medium, notably mammographic image of a high diagnostic quality.

### BRIEF DESCRIPTION OF THE DRAWINGS

These and other aspects of the invention will be elucidated with reference to the embodiments described hereinafter and with reference to the accompanying drawing wherein

FIG. 1 shows a schematic diagram of the apparatus for imaging a turbid medium of the invention;

FIG. 2 shows a schematic cross-sectional drawing of the cup with a breast 1;

FIG. 3 illustrates an implementation of the computation by the analysis module of the optical properties of the breast to be imaged.

### DETAILED DESCRIPTION OF THE EMBODIMENTS

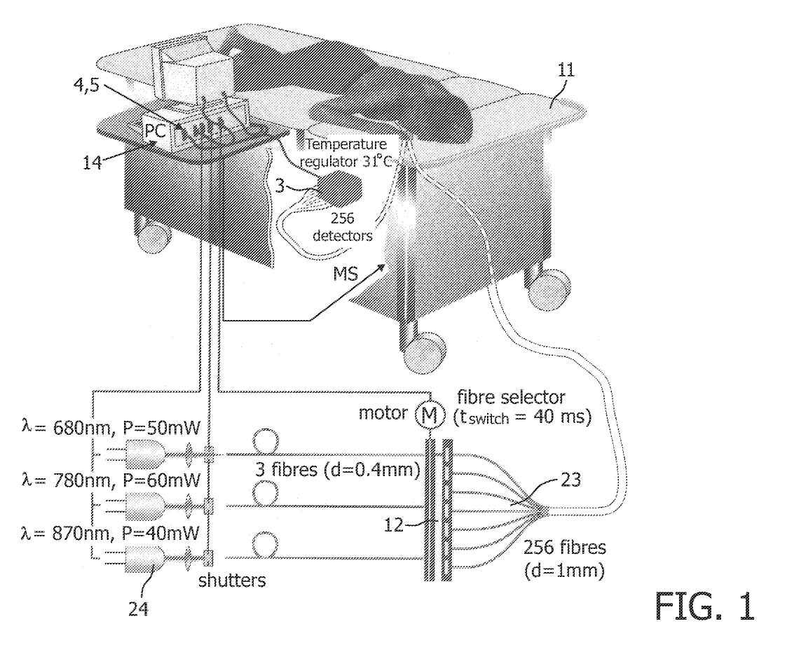

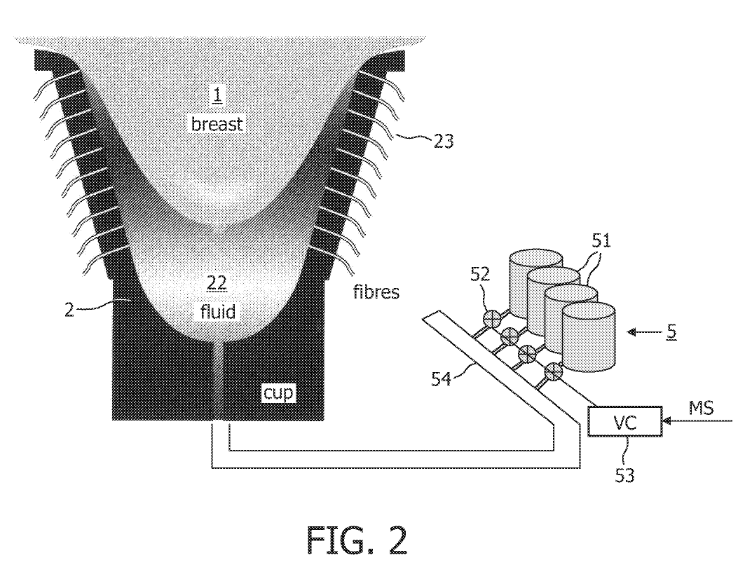

FIG. 1 shows a schematic diagram of the apparatus for imaging a turbid medium of the invention. Notably, the apparatus for imaging a turbid medium shown diagrammatically in FIG. 1 is an optical mammography system. The optical mammography system comprises a carrier 11 on which the patient to be examined (notably a woman whose breast(s) 1 are to be examined) is placed in prone position (i.e. face down) having one breast suspended in the examination space 2 that has to form of a measurement cup 2 (see FIG. 2). The space between the breast 1 and the cup surface is filled with a matching fluid 22, of which optical scattering and absorption properties for example closely match the average absorption and scattering properties of the breast so that transitions of optical properties between the breast tissue and space outside the breast are reduced.

FIG. 2 shows a schematic cross-sectional drawing of the cup with a breast 1. A large number of fibres 23 (510 in total) is connected with one end to the cup. Half of the fibres are connected to detector modules 5 with the other end, and half of the fibres are connected to a fibre-switch 12 with the other end. The fibre-switch 12 can direct light from three different lasers 24 in either one of the 256 source fibres 23 (255 to the cup, one directly to a detector fibre). In this way, either one of the source fibres 23 can provide a conical light beam in the cup. By properly switching the fibre-switch 12, all the source fibres will emit a conical light beam subsequently.

The light from the selected source fibre is scattered by the matching fluid and the breast, and is detected by the 255 detector modules. The detector fibres are often arranged to arrive at detection position in respective rings around the wall of the cup. The rings are displaced along the long axis (usually vertical) of the cup. The scattering of light in breast tissue is strong, which means that only a limited amount of photons can transverse the breast, compared to the reflected (or backscattered) light. Therefore, a large dynamical range should be covered by the detectors (about 9 orders of magnitude). Photodiodes are used as photosensors 5 in the detector modules. The front-end detector electronics includes one of these photodiodes and an amplifier 31. The amplification factor of the amplifier can be switched between several values. The machine first measures at the lowest amplification, and increases the amplification if necessary. The detectors are controlled by a computer 14.

The computer 14 also controls the lasers, the fibre-switch, and the fluid dispensing system. The computer, cup, fibres, detectors, fibre-switch, and the lasers are all mounted into a bed as shown in FIG. 2.

The detected radiation levels, from the detectors are fed to the computer 14, together with the position of the respective detectors. The computer 14 is provided with software to enable the computer to assign the detected radiation levels to the central radiation component and the boundary radiation component, respectively on the basis of the position where the radiation level is detected. The position where the radiation level is detected is directly connected to the position at which the detected radiation leaves the object. Then the computer with appropriate software computes the optical properties, notably the optical absorption and optical scattering of the breast tissue of the woman's breast under examination from the detection levels of the central radiation component and the boundary radiation component. The computer 14 is provided with an analysis module 5 and a reconstruction module 4. These modules are generally implemented in software. From these actual values of the optical properties of the woman's breast, the reference dataset is calculated by the analysis module 5 on the basis of the actual values of the optical properties. That is, in fact a reference measurement is simulated for a homogeneous object having these actual optical properties. From this simulated reference measurement the reference dataset is formed.

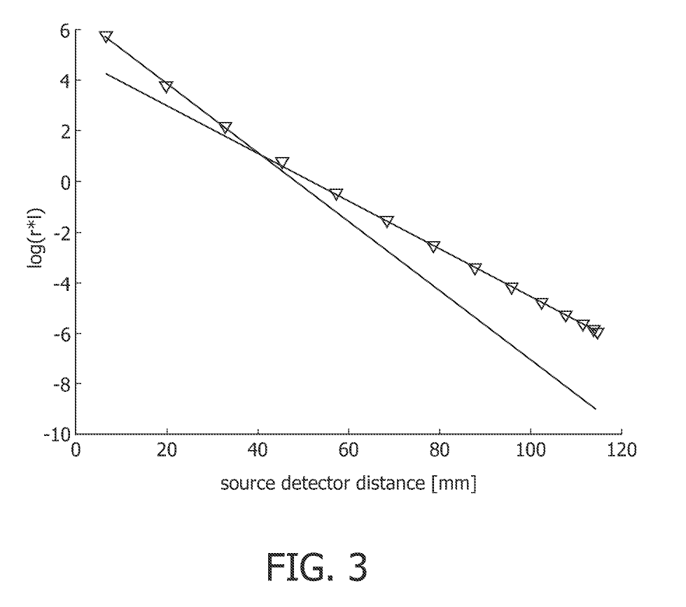

An implementation of the computation by the analysis module 5 of the optical properties of the breast to be imaged is illustrated in FIG. 3. More in particular, optical absorption and optical scattering for a homogeneous object are derived independently by comparing data, i.e. detection levels, where the near-infrared radiation has traveled mostly close to the boundary of the breast with data where the near-infrared radiation has traveled mostly through the inner part of the breast. The analysis module performs the following computations. The data are averaged over the rotational symmetry and/or the symmetry for reflections. Then these averaged data (I) are plotted (see example in FIG. 3) in a log(rI) over r graph. Then the data at source detector distances less than, or larger than the separation value are fitted to linear dependences, respectively. This is illustrated by the two straight drawn lines in the graph of FIG. 3. From the two resulting slopes the analysis module calculates the mean optical scattering coefficient and the mean optical absorption coefficient. In a more refined implementation this approach is carried-out for each ring of detectors, which yields mean optical scattering and absorption coefficients that may vary from ring to ring of detection positions. This is especially important for cases where the size of the breast is significantly smaller than the size of the cup. In this case the data from the lower rings represent the fluid properties and the top ring data represent the breast properties. Averaging the data reduces the effects of inhomogeneities in the breast tissue and increases the signal-to-noise ratio of the data that are employed to derive values of the optical properties. Accordingly, the accuracy is improved of the derived values of the optical properties. Alternatively, the symmetry properties may be employed to reduce the amount of data and accordingly reduces the time required for acquiring data.

Subsequently, on the basis of the mean optical scattering and absorption of coefficients the analysis module forms a reference dataset for a homogeneous object that has the mean optical scattering and mean optical absorption coefficients of the breast to be imaged. That is, in fact the analysis module simulates a reference measurement for a homogeneous object that has the mean optical scattering and absorption properties, This simulation leads to the reference dataset. The reference dataset and the measurement datasets are employed by the reconstruction module which applies a Rytov approximation to reconstruct the interior of the object being imaged. This reconstruction is provided in the form of a volumetric image dataset. More in particular the computation by the reconstruction module 4 is as follows. Image reconstruction is performed using the Rytov approximation, where a reference dataset Φ₀ and a measurement dataset Φ are used to reconstruct an image of the difference Δμ between reference and measurement object:

$$ln \frac{{Φ}^{x} ( s , d )}{{Φ}_{0}^{x} ( s , d )} = - \frac{{κ}_{0}^{2}}{{μ}_{a}} \int {d}^{3} r {Δμ}_{a} ( r ) \frac{{G}_{x} ( r , {r}_{s} ) {G}_{x} ( r , {r}_{d} )}{{G}_{x} ( {r}_{d} , {r}_{s} )}$$

Using the ratio of Φ and Φ₀ has the advantage that system depending calibration factors cancel. However, the Rytov approximation is a first order approximation and is a fair approximation for small changes Δμ.

Typically, the reference measurement is simulated for a homogeneous scattering fluid and the object measurement on a woman's breast. If there is a mismatch between the optical properties of the breast and the fluid, which is usually the case, the Rytov approximation breaks down, because the breast can not be regarded as a small perturbation. In this case the image quality obtained with the Rytov approximation will be poor. The analysis module 5 corrects the reference dataset in such a way as if it was measured with a fluid having the average optical properties of the breast. The correction has three steps:

Estimate the average optical properties of the breast

Simulate a reference measurement for the breast parameters S^{breast}. (If the system is rotationally symmetric the symmetry can be exploited to speed up the calculations significantly.)

Calculate the corrected reference measurement: Φ₀^{c}=Φ₀*S^{breast}/S^{fluid}. Here, S^{fluid} is a simulated reference measurement for the parameters of the fluid. In this way Φ₀^{c} still contains the calibration information of Φ₀.

By using the corrected reference data in the Rytov approximation, the image quality of the breast is improved substantially.

Again with reference to FIGS. 1 and 2, the aspect of preparation of the matching fluid is discussed. On the basis of the optical properties computed by the analysis module 5, i.e. the mean optical scattering and absorption coefficients, the analysis module generates a mixing signal. The mixing signal (MS) represents the optical properties, notably as to optical absorption and scattering, for the matching fluid to closely resemble the mean optical properties of the breast to be imaged. For example, the optical properties of the breast to be imaged can be represented by mixing ratios of basic fluids, such that the fluid mixture has optical properties that accurately correspond to the optical properties of the breast to be imaged. Accordingly, the mixing signal represents mixing ratio's of a number of basic fluids to be mixed into a mixture that has the optical properties of the breast to be imaged. Alternatively, the mixing signal represents the concentration of dye(s) to be added to a basic fluid to achieve the optical properties of the breast to be imaged.

The optical imaging system shown in the example of FIG. 2 is provided with a fluid dispensing system 5. The fluid dispensing system comprises several containers 51 to contain respective different basic fluids. The containers 51 are each with a valve 52 coupled to a supply duct 54 which feeds into the cup 2. The valves 52 are controlled by a valve controller 53. The valve controller 53 is coupled to the analysis unit 4 to receive the mixing signal. Hence, the valve controller operates the valves so that a mixture of basic fluids is formed in the duct, or one of the basic fluids is selected and fed into the cup 2 with the optical properties that closely resemble the optical properties of the breast 1 to be imaged.

1. An imaging system for imaging of a turbid medium (1) comprising
a radiation source (24) to illuminate an object to be imaged,
a detection system (3) to detect radiation from the object to produce a plurality of detected radiation levels at respective positions relative to the object
the radiation from the object having
a central radiation component having passed mainly through an inner region of the object and
a boundary radiation component having passed mainly through a boundary region of the object,
the imaging system further comprising
an analysis module (5) to derive optical properties of the object on the basis of a comparison of the central radiation component and the boundary radiation component and
a reconstruction module (4) to
access the detected radiation levels and the optical properties and
reconstruct an image dataset on the basis of the detected radiation levels and the optical properties.

2. An imaging system for imaging a turbid medium as claimed in claim 1, wherein the reconstruction module is arranged
produce a measurement dataset that represents the detected radiation levels
to derive a reference dataset on the basis of the derived optical properties corresponding to a homogeneous object having the derived optical properties and
to reconstruct the image dataset from the measurement dataset and the reference dataset.

3. An imaging system as claimed in claim 1, wherein
the radiation source is arranged to illuminate the object at a source position
the detection system is arranged to detect radiation from the object at a plurality of detection positions,
the analysis module is arranged
to form a dependence of the detected radiation levels on the distance between the source position and the detection position,
to access a set of dependences of simulated radiation levels on the distance between the source position and the detection position for respective pre-determined values of the optical properties and
to derive the optical properties of the object on the basis of the comparison between the dependencies of the detected radiation levels and the simulated radiation levels on the distance between the source position and the detection position.

4. An imaging system for imaging a turbid medium as claimed in claim 1, wherein
the radiation source is arranged to illuminate the object at a source position
the detection system is arranged
to detect radiation from the object at a plurality of detection positions
to assign to the central radiation component detected radiation levels for detection positions that have a distance from the source position larger than a separation value and
the analysis module is arranged to assign to the boundary radiation component detected radiation levels for detection positions that have a distance from the source position less than a separation value.

5. An imaging system for imaging a turbid medium as claimed in claim 4, wherein the separation value is (i) set by the user or (ii) derived from the dependence of the detected radiation levels on the distance between the source and detection position.

6. An imaging system for imaging a turbid medium as claimed in claim 1, comprising
a receptacle (2) to receive the object (1) to be imaged and
a fluid dispensing system (5)
to prepare a matching fluid on the basis of the derived optical properties and
provide the prepared matching fluid to the receptacle.

7. An imaging system as claimed in claim 2, wherein the reconstruction module is arranged to reconstruct the image on the basis of ratios of respective data values of the measurement dataset and the reference dataset.

8. A method of imaging a turbid medium in which
a central radiation component is identified of radiation having passed mainly through an inner region of the object and
a boundary radiation component is identified of radiation having passed mainly through a boundary region of the object,
optical properties are derived of the object on the basis of a comparison of the central radiation component and the boundary radiation component and
an image dataset is reconstructed on the basis of the detected radiation levels and the optical properties.

9. A computer programme comprising instructions to
to identify a central radiation component of radiation having passed mainly through an inner region of the object and
to identify a boundary radiation component of radiation having passed mainly through a boundary region of the object,
derive optical properties of the object on the basis of a comparison of the central radiation component and the boundary radiation component and
reconstruct an image dataset on the basis of the detected radiation levels and the optical properties.

10. An assessment combination of a detection system (3) and an analysis module (5),
the detection system (3) having the function to detect radiation from the object to produce a plurality of detected radiation levels at respective positions relative to the object
the radiation from the object having
a central radiation component having passed mainly through an inner region of the object and
a boundary radiation component having passed mainly through a boundary region of the object,
the analysis module (5) having the function to derive optical properties of the object on the basis of a comparison of the central radiation component and the boundary radiation component.
